(19) United States
(12) Patent Application Publication
(10) Pub. No.: US 2007/0235333 A1
(43) Pub. Date: Oct. 11, 2007

(54) Patterned chip with adjustable surface and method for manufacturing the same

(76) Inventors: Peilin Chen, Taipei (TW); Jau-Ye Shiu, Taipei (TW); Chiung-Wen Kuo, Taipei (TW)

Correspondence Address: J.C. Patents, SUITE 250, 4 VENTURE, IRVINE, CA, 92618, US

(21) Appl. No.: 11/400,507

(22) Filed: Apr. 7, 2006

(51) Int. Cl. C07K 1/26

(52) U.S. Cl. 204/450

(57) ABSTRACT

The invention is to provide a method for fabricating a patterned chip with adjustable surface including of a basic layer, an insulating layer and a layer of biomolecules or cells. The method comprises of the following steps: pre-patterning a basic layer by photolithography; coating the pre-patterned basic layer with an insulating layer, the insulating layer is placed in solution containing biomolecules or cells with external voltage applied to the insulating layer. In addition, two steps—fabricating an pattern on the insulating layer matching the pattern of the basic layer by photolithography and treating the patterned insulating surface with chemical, physical or plasma surface process treatment—can be further followed by the step of pre-patterning a basic layer by photolithography, then proceeding the remaining steps.

### FIELD OF THE INVENTION

The present invention relates to a method of fabricating a patterned chip, and more particularly, to a method of manufacturing a patterned chip with adjustable surface and the state of the adjustable surface can be switched from hydrophobic to hydrophilic state.

### BACKGROUND OF THE INVENTION

U.S. Pat. No. 6,911,132 B2 disclosed that the apparatus could transport and manufacture a drop by electro wetting to another portion of the chip. However, a super hydrophobic surface was resistant to the adsorption of any hydrophilic materials, for example, cells or biomolecules did not stick or adhere on the super hydrophobic surfaces. So, although the electro wetting-based prior art was useful in certain applications, users had to manipulate drops to transport to other detecting portions, which is limited in convenience and simplicity. In particular, no prior arts provided a mechanism for patterning and immobilizing hydrophilic materials on adjustable surface for biological diagnosis.

According to the foregoing disadvantages, the invention is to provide a novel surface-manipulative technique to manufacture adjustable surface and its application for biological diagnosis.

### SUMMARY OF THE INVENTION

An object of the invention is to provide a method for fabricating a patterned chip with adjustable surface, comprising of:
(a) pre-patterning a basic layer by photolithography;
(b) coating the pre-patterned basic layer with an insulating layer;
(c) the insulating layer is placed in solution containing biomolecules or cells, with external voltage between 0 and 300 Volt applied to the insulating layer. The insulating surface can be switched from hydrophobic to hydrophilic state, even from super hydrophobic surface to super hydrophilic state, thereof to immobilize the biomolecules or cells on the insulating layer.

Another object of this invention is to provide a method for fabricating a patterned chip with adjustable surface, comprising:
(a) pre-patterning a basic layer by photolithography;
(b) coating the pre-patterned basic layer with an insulating layer,
(c) fabricating an pattern on the insulating layer matching the pattern of the basic layer by photolithography;
(d) treating the insulating layer with chemical, physical or plasma surface process treatment, the insulating layer being the modified layer; and
(e) the modified layer is placed in solution containing biomolecules or cells, with external voltage between 0 and 300 Volt applied to the modified layer. The insulating surface can be switched from hydrophobic to hydrophilic state, even from super hydrophobic surface to super hydrophilic state, further from contact angle more than 150 degree to contact angle less than 10 degree, thereof to immobilize the biomolecules or cells on the insulating layer.

Another object of this invention is to provide a patterned chip with adjustable surface for bio-diagnosis including a basic layer, an insulating layer coated on the basic layer, and a layer of biomolecules or cells immobilized on the insulating layer.

Another object of this invention is to provide a patterned chip with adjustable surface for bio-diagnosis including a basic layer, an insulating layer coated on the basic layer, and a layer of biomolecules or cells immobilized on the insulating layer, wherein the patterned array of the chip is addressable to perform desired tasks; selectively, the insulating layer further could be roughened by physical, chemical or plasma surface treatment in advance.

In order to achieve the above objects, the method for fabricating a patterned chip with adjustable surface, comprising of the following steps:
(a) pre-patterning a basic layer by photolithography: a layer of photoresist is applied on the top of the basic layer, desired areas of photoresist to be removed are exposed to light, photoresist is developed to remove the exposed photoresist, the bare windows (the materials without the protection of photoresist) are etched by etching solution, and then a basic layer is pre-patterned, wherein other types of resist sensitive to X-rays or sensitive to electron-beam exposure may be used; alternatively, a layer of photoresist is on the top of the basic layer, desired areas of photoresist to be remained are exposed to light, photoresist are developed to remove the unexposed photoresist, the bare windows are etched by etching solution, and a basic layer is pre-patterned, wherein other types of resist sensitive to X-rays or sensitive to electron-beam exposure may be used;
(b) coating the pre-patterned basic layer with an insulating layer;
(c) the insulating layer is placed in solution containing biomolecules or cells, with external voltage applied to the insulating layer: when the external voltage is applied to the insulating layer, the state of the insulating layer thereof switch from hydrophobic to hydrophilic state, even from super hydrophobic state to super hydrophilic state, conducting hydrophilic materials such as biomolecules or cells to adhere or stick on the patterned area where the underneath pre-patterned basic layer is activated by external voltage; the external voltage can be between 0 and 300 Volt.

Selectively, two steps can be further added between step (b) and step (c), including: fabricating an pattern on the insulating layer matching the pattern of the basic layer by photolithography, and treating the patterned insulating surface with chemical, physical or plasma surface process treatment—a layer of photoresist is applied on the top of the insulating layer, desired areas of photoresist to be removed are exposed to light, photoresist are developed to remove the exposed photoresist, and the bare windows (the materials without the protection of photoresist) are for chemical, physical or plasma surface process treatment, with the insulating layer being the modified layer, wherein other types of resist sensitive to X-rays or sensitive to electron-beam exposure may be used; alternatively, a layer of photoresist is on the top of the insulating layer, desired areas of photoresist to be remained are exposed to light, photoresist is developed to remove the unexposed photoresist, and the bare windows are for chemical, physical or plasma surface process treatment, with the insulating layer being the modified layer, wherein other types of resist sensitive to X-rays or sensitive to electron-beam exposure may be used.

Therefore, the hydrophilic materials such as biomolecules or cells immobilized and patterned on the surface of the insulating layer enable analysis of the level of their recognizable targets, with typically using the reaction of an antibody or antibodies to its antigen, but not limited.

The layer of biomolecules or cells is generated in the form of arrays that are immobilized on a solid support, and is used to probe the specific binding pair member for those having certain defined characteristics.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 is an experimental data diagram of the contact angle of the modified layer of the preferred embodiment according to the invention;

FIG. 2 is an experimental data diagram of the contact angle of the modified layer with external voltage applied of the preferred embodiment according to the invention;

FIG. 3A is an image of a drop sitting on the modified layer of the preferred embodiment according to the invention;

FIG. 3B is an image of a drop sitting on the modified layer of the preferred embodiment according to the invention;

FIG. 4 is a sequential schematic view of the method for fabricating a patterned chip of the preferred embodiment according to the invention;

FIG. 5 is a cross-sectional view of the chip structure of the preferred embodiment according to the invention;

FIG. 6 is an optical image of the modified surface of the first preferred embodiment according to the invention;

FIG. 7 is a fluorescence image of FITC conjugated anti-chicken IgG dipped into the modified surface with 150 V applied for 1 second of the first preferred embodiment according to the invention;

FIG. 8 is a fluorescence image of the cy3-conjugated anti-rabbit IgG dipped into the modified surface with 150 V applied for 1 second of the second preferred embodiment according to the invention;

FIG. 9 is a fluorescence image of the TRITC conjugated anti-goat IgG bonded to pre-patterned goat IgG dipped into the modified surface of the third preferred embodiment according to the invention;

FIG. 10 is a fluorescence image of the multi-component proteins dipped into the modified surface of the fourth preferred embodiment according to the invention;

FIG. 11 is an optical image of patterned HeLa cells dipped into the modified surface of the fifth preferred embodiment according to the invention;

FIG. 12 is a fluorescence image of labeled HeLa cells dipped into the modified surface of the fifth preferred embodiment according to the invention.

### DESCRIPTION OF THE PREFERRED EMBODIMENTS

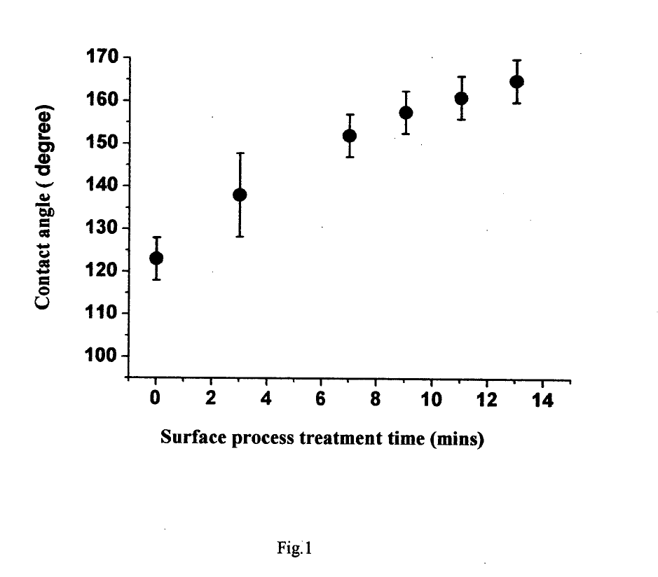

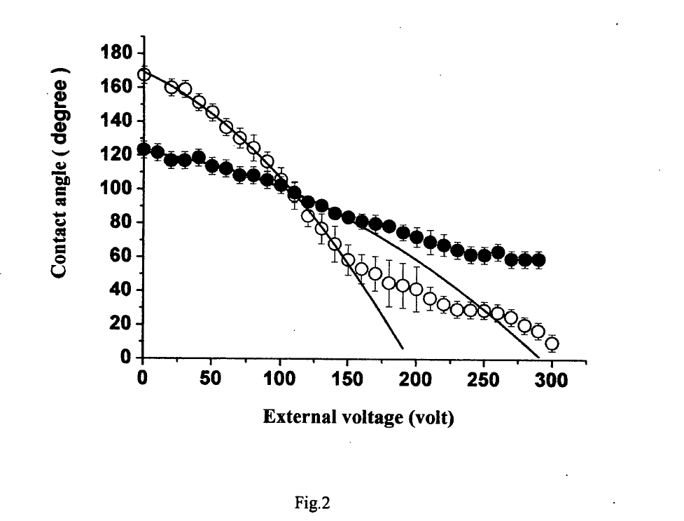

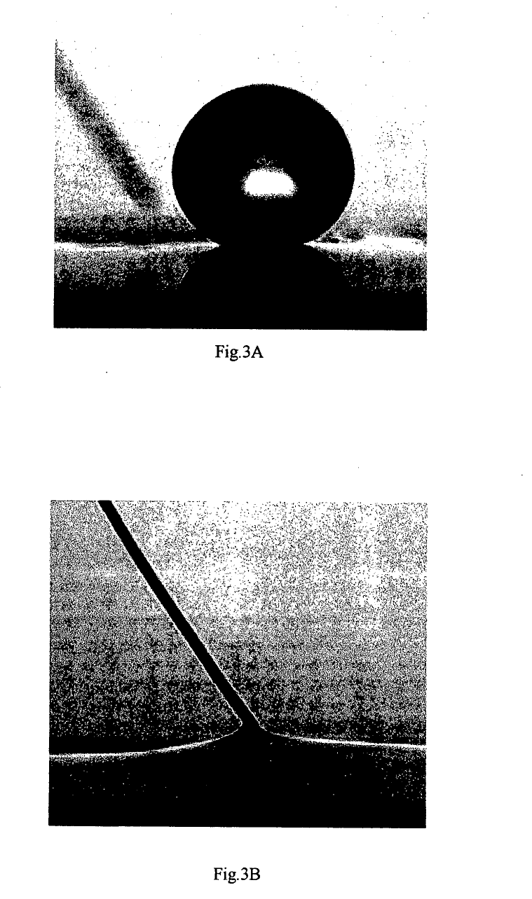

Before going to precede several preferred embodiments exemplified as herein, we display a method of getting adjustable (super) hydrophilic surface, comprising of the following steps:
a. Coating a basic layer with an insulating layer: Teflon AF (DuPont) layer as the insulating layer is coated on ITO (Indium Tin Oxide) electrode as the basic layer by the method of spin coating;
b. treating the insulating surface with chemical, physical or plasma surface process treatment, with the insulating layer being the modified layer: the Teflon AF (DuPont) layer is treated with oxygen-containing plasma to have rough surface. As illustrated in FIG. 1, the contact angle of Teflon AF (DuPont) for liquids increases with the oxygen plasma treatment time increasing, the Teflon AF (DuPont) layer becoming more hydrophobic;
c. applying an external voltage to the modified layer: referred to FIG. 2, the full circle represents the relation of the contact angles of the unmodified insulating surface for liquids to the external voltage, and the hollow circle expresses the relation of the contact angles of the modified insulating surface for liquids to the external voltage, wherein the external voltage is between 0 and 300 Volt. Knowledge from FIG. 2 is that the contact angle of the insulating surface for liquids decreases with the value of the voltages increasing, and the modified insulating surface can be switched from hydrophobic to hydrophilic state, even from super hydrophobic surface to super hydrophilic state, further from contact angle more than 150 degree to contact angle less than 10 degree. The hydrophobic state of the insulating surface can be illustrated in FIG. 3A, while the hydrophilic state in FIG. 3B.

Taken in conjunction with the above-mentioned features, the objects, features and advantages of the present invention will be more readily understood upon consideration description of the following detailed first, second, third, fourth and fifth preferred embodiments related to the invention.

For purposes of the present embodiment disclosure, the term “pattern” is used herein to indicate to fabricate a matrix array, unless it has other meanings to identify itself. For example, the sentence of “pre-pattern a basic layer by photolithography” means to create a matrix array on a basic layer by photolithography.

### First Preferred Embodiment

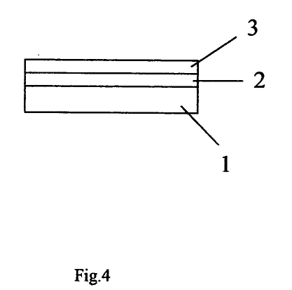

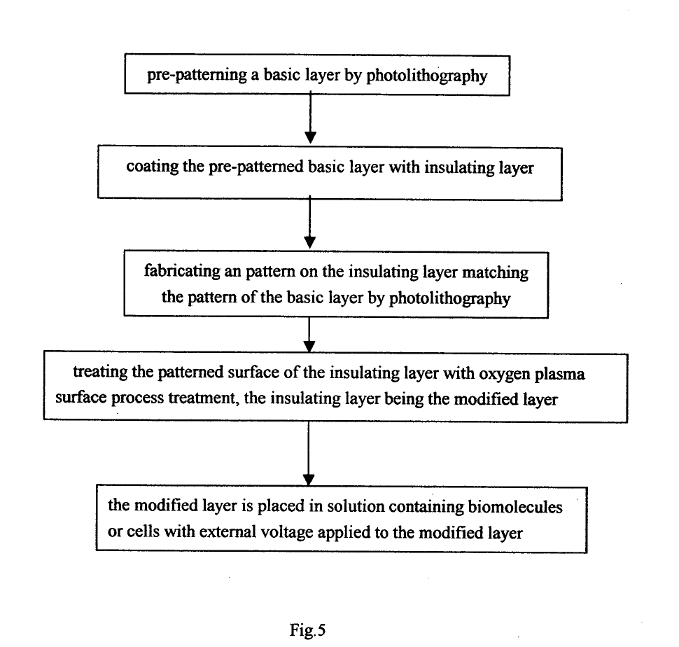

With reference to FIG. 4 and FIG. 5, FIG. 4 is a sequential schematic view of the method for fabricating a patterned chip of the preferred embodiment according to the invention; FIG. 5 is a cross-sectional view of the chip structure of the preferred embodiment according to the invention.

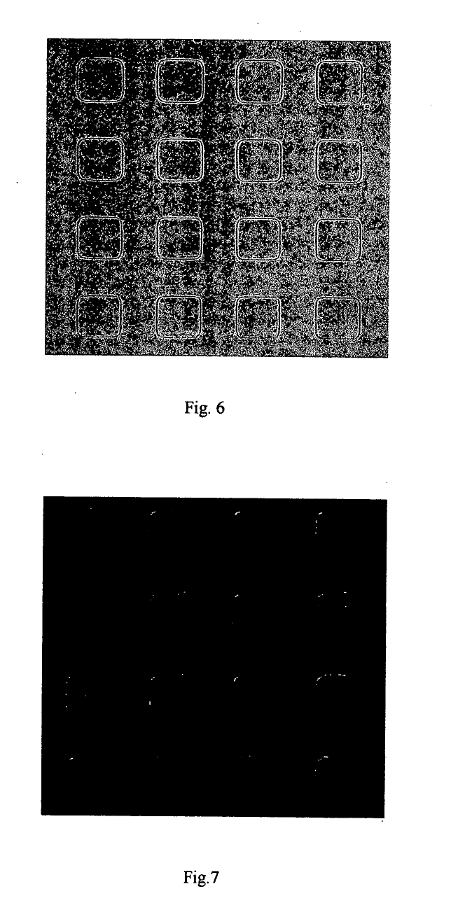

A method for fabricating a patterned chip with adjustable surface including a basic layer, an insulating layer and a layer of biomolecules or cells, comprising of the following steps:
(a) pre-patterning a basic layer by photolithography: a layer of S1813 photoresist is applied on the top of the ITO basic layer, desired areas of photoresist to be removed are exposed to light, photoresist are developed to remove the exposed photoresist by M319 developer, the bare windows (the materials without the protection of photoresist) are etched by Chlorazotic acid, the remained photoresist are washed away by acetone, then a basic layer is pre-patterned, wherein other types of resist sensitive to X-rays or sensitive to electron-beam exposure may be used;
(b) coating the pre-patterned basic layer with insulating layer: Teflon AF (DuPont) layer as the insulating layer is coated on the ITO basic layer by the method of spin-coating;
(c) fabricating an pattern on the insulating layer matching the pattern of the basic layer by photolithography: a layer of S1813 photoresist is applied on the top of the insulating layer, desired areas of photoresist to be removed are exposed to light, photoresist are developed to remove the exposed photoresist by MF319 developer, and the bare windows (the materials without the protection of photoresist) are for the following step, wherein other types of resist sensitive to X-rays or sensitive to electron-beam exposure may be used;
(d) treating the patterned surface of the insulating layer with oxygen plasma surface process treatment, the insulating layer being the modified layer: referred to FIG. 6, it shows an optical image of the modified surface of the first preferred embodiment according to the invention;
(e) the modified layer is placed in solution containing biomolecules or cells with external voltage applied to the modified layer: the modified Teflon AF (DuPont) layer is placed in PBS solution containing FITC-conjugated anti-chicken IgG with external voltage 150 V applied for 1 second. The state of the modified Teflon AF (DuPont) layer is switched from hydrophobic to hydrophilic state, even from super hydrophobic state to super hydrophilic state, conducting FITC conjugated anti-chicken IgG to adhere or stick on the patterned area where the underneath pre-patterned basic layer are activated by external voltage. FIG. 7 shows a fluorescence image of FITC conjugated anti-chicken IgG dipped into the modified surface with 150 V applied for 1 second. The modified surface becomes hydrophilic without voltage applied as long as the biomolecules stayed on the surface.

The FITC conjugated anti-chicken IgG immobilized on the surface of the insulating layer are to enable analysis of the level of substances capable of binding to the FITC conjugated anti-chicken IgG, by using the reaction of an antibody or antibodies to its antigen.

### Second Embodiment

We still provide a second embodiment to show that another kind of antigens “anti-rabbit IgG” will be immobilized on the super hydrophobic surface with voltage applied to.

With reference to FIG. 4 and FIG. 5, FIG. 4 is a sequential schematic view of the method for fabricating a patterned chip of the preferred embodiment according to the invention; FIG. 5 is a cross-sectional view of the chip structure of the preferred embodiment according to the invention.

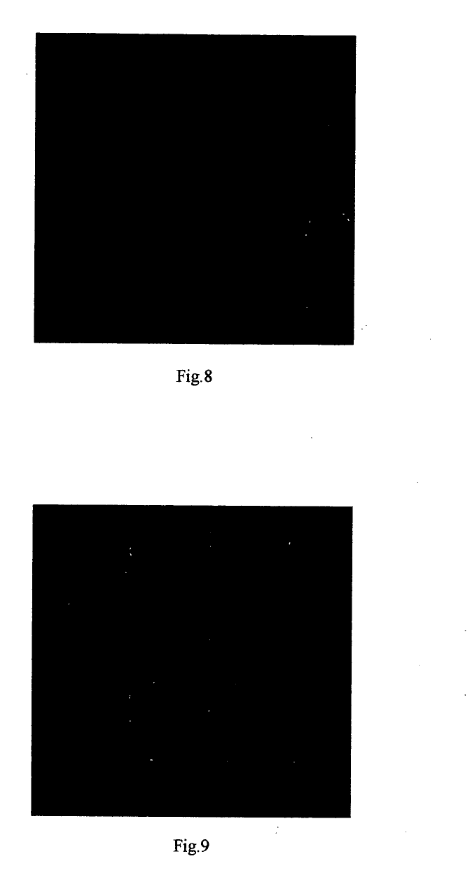

A method for fabricating a patterned chip with adjustable surface for biological diagnosis including a basic layer, an insulating layer and a layer of biomolecules or cells, comprising of the following steps:
(a) pre-patterning a basic layer by photolithography: a layer of S1813 photoresist is coated on the top of the ITO basic layer, desired areas of photoresist to be removed are exposed to light, photoresist are developed to remove the exposed photoresist by MF319 developer, the bare windows (the materials without the protection of photoresist) are etched by Chlorazotic acid, the remained photoresist are washed away by acetone, and then the basic layer is pre-patterned, wherein other types of resist sensitive to X-rays or sensitive to electron-beam exposure may be used,
(b) coating the pre-patterned basic layer with insulating layer: Teflon AF (DuPont) layer as the insulating layer is coated on the ITO basic layer by the method of spin-coating;
(c) fabricating an pattern on the insulating layer matching the pattern of the basic layer by photolithography: a layer of S1813 photoresist is applied on the top of the insulating layer, desired areas of photoresist to be removed are exposed to light, photoresist is developed to remove the exposed photoresist by MF319 developer, and the bare windows (the materials without the protection of photoresist) are for the following step, wherein other types of resist sensitive to X-rays or sensitive to electron-beam exposure may be used;
(d) treating the patterned surface of the insulating layer with oxygen plasma surface process treatment, the insulating layer being the modified layer;
(e) the modified layer is placed in solution containing biomolecules or cells with external voltage applied to the modified layer: the modified Teflon AF (DuPont) layer is placed in a PBS solution containing cy3 conjugated anti-rabbit IgG with an external voltage 150 V applied for 1 second. The state of the modified Teflon AF (DuPont) layer is switched from hydrophobic to hydrophilic state, even from super hydrophobic state to super hydrophilic state, conducting cy3 conjugated anti-rabbit IgG to adhere or stick on the patterned area where the underneath pre-patterned basic layer are activated by external voltage. FIG. 8 is a fluorescence image of the cy3-conjugated anti-rabbit IgG dipped into the modified surface with 150 V applied for 1 second of the second preferred embodiment according to the invention. The modified surface becomes hydrophilic without voltage applied as long as the biomolecules stayed on the surface.

The cy3 conjugated anti-rabbit IgG immobilized on the surface of the insulating layer are to enable analysis of the level of substances capable of binding to the cy3 conjugated anti-rabbit IgG, by using the reaction of an antibody or antibodies to its antigen.

### Third Embodiment

To demonstrate that the protein array produced by such process still retain its activity, the third preferred embodiment is provided.

With reference to FIG. 4 and FIG. 5, FIG. 4 is a sequential schematic view of the method for fabricating a patterned chip of the preferred embodiment according to the invention; FIG. 5 is a cross-sectional view of the chip structure of the preferred embodiment according to the invention.

A method for fabricating a patterned chip with adjustable surface for biological diagnosis including a basic layer, an insulating layer and a layer of biomolecules or cells, comprising of the following steps:
(a) pre-patterning a basic layer by photolithography: a layer of S1813 photoresist is applied on the top of the ITO basic layer, desired areas of photoresist to be removed are exposed to light, photoresist is developed to remove the exposed photoresist by MF319 developer, the bare windows (the materials without the protection of photoresist) are etched by Chlorazotic acid, the remained photoresist is washed away by acetone, and then a basic layer is pre-patterned, wherein other types of resist sensitive to X-rays or sensitive to electron-beam exposure may be used;
(b) coating the pre-patterned basic layer with insulating layer: Teflon AF (DuPont) layer as the insulating layer is coated on the ITO basic layer by the method of spin-coating;
(c) fabricating an pattern on the insulating layer matching the pattern of the basic layer by photolithography: a layer of S1813 photoresist is applied on the top of the insulating layer, desired areas of photoresist to be removed are exposed to light, photoresist is developed to remove the exposed photoresist by MF319 developer, and the bare windows (the materials without the protection of photoresist) are for the following step, wherein other types of resist sensitive to X-rays or sensitive to electron-beam exposure may be used;
(d) treating the patterned surface of the insulating layer with oxygen plasma surface process treatment, the insulating layer being the modified layer:
(e) the modified layer is placed in solution containing biomolecules or cells with external voltage applied to the modified layer: the modified Teflon AF (DuPont) layer is placed in a PBS solution containing goat IgG with external voltage 150 V applied for 1 second, and the state of the modified Teflon AF (DuPont) layer switch from hydrophobic to hydrophilic state, even from super hydrophobic state to super hydrophilic state, conducting goat IgG to adhere or stick on the patterned areas where the underneath pre-patterned basic layer are activated by external voltage. The modified surface becomes hydrophilic without applying voltage as long as the biomolecules stayed on the surface; and
   Additionally,
(f) placing the whole chip in the solutions containing whatever is specific to said biomolecules: FIG. 9, a fluorescence image of the TRITC conjugated anti-goat IgG bonded to pre-patterned goat IgG dipped into the modified surface, showing that the protein array produced by such process still retain its activity.

### Fourth Embodiment

Moreover, a chip of the embodiment in accordance with the invention having an array of multi-component proteins is possible.

With reference to FIG. 4 and FIG. 5, FIG. 4 is a sequential schematic view of the method for fabricating a patterned chip of the preferred embodiment according to the invention; FIG. 5 is a cross-sectional view of the chip structure of the preferred embodiment according to the invention.

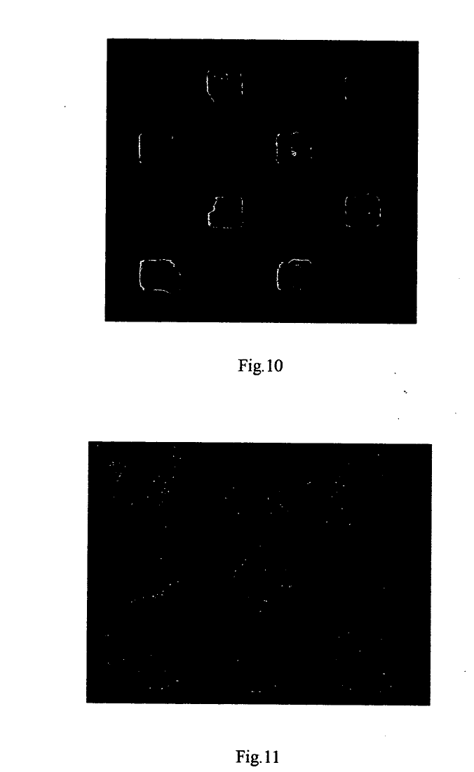

A method for fabricating a patterned chip with adjustable surface for biological diagnosis including a basic layer, an insulating layer and a layer of biomolecules or cells, comprising of the following steps:
(a) pre-patterning a basic layer by photolithography: a layer of S1813 photoresist is applied on the top of the ITO basic layer, desired areas of photoresist to be removed are exposed to light, photoresist is developed to remove the exposed photoresist by MF319 developer, the bare windows (the materials without the protection of photoresist) are etched by Chlorazotic acid, the remained photoresist is washed away by acetone, and then a basic layer is pre-patterned, wherein other types of resist sensitive to X-rays or sensitive to electron-beam exposure may be used;
(b) coating the pre-patterned basic layer with insulating layer: Teflon AF (DuPont) layer as the insulating layer is coated on the ITO basic layer by the method of spin coating;
(c) fabricating an pattern on the insulating layer matching the pattern of the basic layer by photolithography: a layer of S1813 photoresist is applied on the top of the insulating layer, desired areas of photoresist to be removed are exposed to light, photoresist is developed to remove the exposed photoresist by MF319 developer, and the bare windows (the materials without the protection of photoresist) are for the following step, wherein other types of resist sensitive to X-rays or sensitive to electron-beam exposure may be used;
(d) treating the patterned surface of the insulating layer with oxygen plasma surface process treatment, the insulating layer being the modified layer:
(e) the modified layer is placed in solution containing biomolecules or cells with external voltage applied to the modified layer: the modified Teflon AF (DuPont) layer is placed in a PBS solution containing FITC conjugated anti-chicken IgG with an external voltage 150 V applied for 1 second, and the state of the modified Teflon AF (DuPont) layer switch from hydrophobic to hydrophilic state, even from super hydrophobic state to super hydrophilic state, conducting. FITC conjugated anti-chicken IgG to adhere or stick on the patterned area where the underneath pre-patterned basic layer are activated by external voltage, wherein the external voltage can be between 0 and 300 Volt; and
   Additionally,
(f) repeating step (e) with different areas of the underneath basic layer being successively activated by external voltages: placing the whole chip in the solution containing Cy3 conjugated anti-rabbit IgG with second external voltage 150 V applied for 1 second at other specific desired areas. With reference with FIG. 10, it is a fluorescence image of the multi-component proteins dipped into the modified surface, wherein Green color is FITC conjugated anti-chicken IgG and red is Cy3 conjugated anti-rabbit IgG.

### Fifth Embodiment

A chip of the embodiment in accordance with the invention having an array of cells may be possible.

With reference to FIG. 4 and FIG. 5, FIG. 4 is a sequential schematic view of the method for fabricating a patterned chip in accordance with the preferred embodiments of the present invention; FIG. 5 is a cross-sectional view of the chip structure in accordance with the preferred embodiments of the present invention.

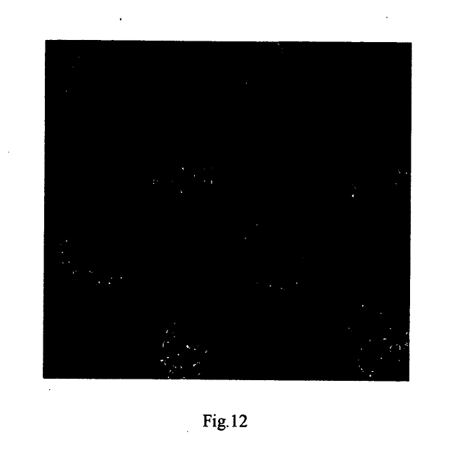

A method for fabricating a patterned chip with adjustable surface for biological diagnosis including a basic layer, an insulating layer and a layer of biomolecules or cells, comprising of the following steps:
(a) pre-patterning a basic layer by photolithography: a layer of S1813 photoresist is applied on the top of the ITO basic layer, desired areas of photoresist to be removed is exposed to light, photoresist is developed to remove the exposed photoresist by MF319 developer, the bare windows (the materials without the protection of photoresist) are etched by Chlorazotic acid, the remained photoresist is washed away by acetone, and then a basic layer is pre-patterned, wherein other types of resist sensitive to X-rays or sensitive to electron-beam exposure may be used;
(b) coating the pre-patterned basic layer with insulating layer: Teflon AF (DuPont) layer as the insulating layer is coated on the ITO basic layer by the method of spin-coating;
(c) fabricating an pattern on the insulating layer matching the pattern of the basic layer by photolithography: a layer of S1813 photoresist is applied on the top of the insulating layer, desired areas of photoresist to be removed are exposed to light, photoresist is developed to remove the exposed photoresist by MF319 developer, and the bare windows (the materials without the protection of photoresist) are for the following step, wherein other types of resist sensitive to X-rays or sensitive to electron-beam exposure may be used;
(d) treating the patterned surface of the insulating layer with oxygen plasma surface process treatment, the insulating layer being the modified layer;
(e) the modified layer is placed in solution containing biomolecules with external voltage applied to the modified layer: the modified Teflon AF (DuPont) layer is placed in fibronectin solution with external voltage 150 V applied for 1 second, and the state of the modified Teflon AF (DuPont) layer is switched from hydrophobic to hydrophilic state, even from super hydrophobic state to super hydrophilic state, conducting fibronectin to adhere or stick on the patterned area where the underneath pre-patterned basic layer are activated by external voltage; and
   Additionally,
(f) placing the whole chip in solutions containing cells: placing the whole device in solutions containing HeLa (Human Cervical Adenocarcinoma) Cells, and HeLa cells are adhered to the top of fibronectin. With reference to FIG. 11 and FIG. 12, they are respectively an optical image of patterned HeLa cells dipped into the modified surface of the fifth preferred embodiment according to the invention, and a fluorescence image of labeled HeLa cells dipped into the modified surface of the fifth preferred embodiment according to the invention.

What is claimed is:

1. A method for fabricating a patterned chip with adjustable surface including a basic layer, an insulating layer and a layer of biomolecules or cells, comprising of the following steps:
(a) pre-patterning a basic layer by photolithography;
(b) coating the pre-patterned basic layer with an insulating layer;
(c) the insulating layer is placed in solution containing biomolecules or cells with external voltage applied to the insulating layer.

2. The method as claim 1, wherein the range of said external voltage is between 0 and 300 Volt.

3. The method as claim 1, wherein the adjustable range of the insulating surface is from contact angle more than 150 degree to contact angle less than 10 degree, with external voltage applied.

4. The method as claim 1, wherein the patterned arrays of the chip are addressable.

5. The method as claim 1, wherein said step (c) is repeated several times, with different areas of the underneath basic layer being successively activated by external voltages.

6. The method as claim 5, wherein said solutions contain different biomolecules or cells from those at each time.

7. The method as claim 1, wherein the basic layer is such silicon compounds as wafer or glass.

8. The method as claim 1, wherein the basic layer is such conducting material as ITO glass.

9. The method as claim 1, wherein the insulating layer is dielectric layer.

10. The method as claim 1, wherein the insulating layer is a layer of polymers.

11. The method as claim 10, wherein the polymers are polymers with low surface energy.

12. The methods as claim 11, wherein the polymers with low surface energy are polyvinyl chloride (PVC), Polystyrene (PS), or Polydimethysiloxane (PDMS).

13. The method as claim 12, wherein the polymers with low surface energy are fluoride polymers.

14. The method as claim 13, wherein the fluoride polymer is Teflon AF (DuPont).

15. The method as claim 1, wherein the coating method of the insulating layer on the basic layer is spin coating, linear coating, or spin plus linear coating.

16. The method as claim 1, wherein said cells are HeLa cells.

17. The method as claim 1, wherein said biomolecules are such as proteins.

18. The method as claim 17, wherein said proteins are such antigens as anti-chicken IgG or anti-rabbit IgG.

19. The method as claim 17, wherein said proteins are such antibodies as goat IgG.

20. The method as claim 1, wherein said photolithography comprises the following steps:
a. applying a layer of photoresist;
b. exposing desired areas to be removed to light;
c. photoresist is developed by exposing it to a developer solution to remove the exposed photoresist.

21. The method as claim 1, wherein said photolithography comprises the following steps:
a. applying a layer of photoresist;
b. exposing desired areas to be remained to light;
c. photoresist are developed by exposing it to a developer solution to remove the unexposed photoresist.

22. The method as claim 1, wherein etching solution is used to etch at least part of the basic layer after photolithography patterned the basic layer.

23. The method as claim 22, wherein the etching solution is such as Chlorazotic acid.

24. A method for fabricating a patterned chip with adjustable surface including a basic layer, an insulating layer and a layer of biomolecules or cells, comprising of the following steps:
(a) pre-patterning a basic layer by photolithography;
(b) depositing an insulating layer on the pre-patterned basic layer;
(c) fabricating an pattern on the insulating layer matching the pattern of the basic layer by photolithography;
(d) treating the patterned surface of the insulating layer with chemical, physical or plasma surface process treatment, the insulating layer being the modified layer; and
(e) the modified layer is placed in solution containing biomolecules or cells, with external voltage applied to the modified layer.

25. The method as claim 24, wherein the range of said external voltage is between 0 and 300 Volt.

26. The method as claim 24, wherein the adjustable range of the modified surface is from contact angle more than 150 degree to contact angle less than 10 degree, with external voltage applied.

27. The method as claim 24, wherein the patterned arrays of the chip are addressable.

28. The method as claim 24, wherein said step (e) can be repeated several times, with different areas of the underneath basic layer being successively activated by external voltages.

29. The method as claim 28, wherein said solutions can contain different biomolecules or cells from that at each time.

30. The method as claim 24, wherein the basic layer can be such silicon compounds as wafer or glass.

31. The method as claim 24, wherein the basic layer can be such conducting materials as ITO glass.

32. The method as claim 24, wherein the insulating layer is a dielectric layer.

33. The method as claim 24, wherein the insulating layer is a layer of polymers.

34. The method as claim 33, wherein the polymers are polymers with low surface energy.

35. The method as claim 34, the polymers with low surface energy are polyvinyl chloride (PVC), Polystyrene (PS), or Polydimethysiloxane (PDMS).

36. The method as claim 35, wherein the polymers with low surface energy can be fluoride polymers.

37. The method as claim 36, wherein the fluoride polymers are Teflon AF (DuPont).

38. The method as claim 24, wherein the coating method of the insulating layer on the basic layer is spin coating, linear coating, or spin plus linear coating.

39. The method as claim 24, wherein said biomolecules are such as proteins.

40. The method as claim 39, wherein said proteins are such antigens as anti-chicken IgG or anti-rabbit IgG.

41. The method as claim 39, wherein said proteins are such antibodies as goat IgG.

42. The method as claim 24, wherein said photolithography comprises the following steps:
a. applying a layer of photoresist;
b. exposing desired areas to be removed to light;
c. photoresist are developed by exposing it to a developer solution to remove the exposed photoresist.

43. The method as claim 24, wherein said photolithography comprises the following steps:
a. applying a layer of photoresist;
b. exposing desired areas to be remained to light;
c. photoresist are developed by exposing it to a developer solution to remove the unexposed photoresist.

44. The method as claim 24, wherein etching solution is used to etch at least part of the basic layer after photolithography patterned the basic layer.

45. The method as claim 44, wherein the etching solution is such as Chlorazotic acid.

46. A patterned chip with an adjustable surface for biological diagnosis comprising a basic layer, an insulating layer coated on the basic layer, and a layer of biomolecules or cells immobilized on the insulating layer, wherein the arrays of said pattern of the chip are addressable by photolithography.

47. The patterned chip as claim 46, wherein said layer of biomolecules or cells is a layer of multi-component proteins or cells.
